(19) United States
(12) Patent Application Publication
(10) Pub. No.: US 2012/0130268 A1
(43) Pub. Date: May 24, 2012

(54) INTEGRATED ABSORBENT UNDERGARMENT THERMAL INDICATOR AND DISCOMFORT SENSORS

(76) Inventor: HESHAM FAYED, Doral, FL (US)

(21) Appl. No.: 12/953,380

(22) Filed: Nov. 23, 2010

(51) Int. Cl. A61B 5/01; A61F 13/42

(52) U.S. Cl. 600/549; 604/361

(57) ABSTRACT

Presented is an absorbent undergarment with the ability to sense urine, feces and continuously monitor and display the body temperature of the user. The novel integrated undergarment also allows for a contact liquid crystal thermochromic indicator to visually display the user's body temperature. The device may further have the ability to relay the body temperature reading via wireless communication to a remote location, such as a monitoring base, a computer, a Bluetooth receiver, or a handheld wireless device.

### CROSS REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

I. Field of the Invention

This invention generally relates to an absorbent undergarment, such as those used by persons at infancy or with lack of bladder and bowel control, with the ability to continuously monitor and display body temperature and body effluents that cause discomfort. More particularly, this invention relates to a temperature measuring device, such as a contact liquid crystal thermochromic indicator, adapted to be integrated in selective sites on baby and adult absorbent undergarments to continuously and visually indicate the baby's or adult's body temperature and sensors to detect and notify about the presence of body effluents such as urine and fecal matter. The invention has the ability to relay the sensors measurements and readings via wireless communication to a remote location, such as a monitoring base, a computer, a Bluetooth receiver, or a handheld wireless device.

II. Description of Related Prior Art

Almost all babies and young children wear absorbent undergarments because of their inability to control certain bodily functions. Adult absorbent undergarments are also frequently worn by incontinent adults. Absorbent undergarments may be made of cloth or produced from synthetic materials, but most fit the user in the same general manner as do standard underwear.

When a child cries it is often difficult to determine the underlying cause. A baby may cry for a multitude of reasons including, but not limited to, a soiled absorbent undergarment, hunger, discomfort, pain, or desire for love. A baby having a fever will not necessarily cry to directly reflect the underlying symptom as it is frequently a fever associated symptom that causes a baby to cry. High fever could potentially be very dangerous in an infant and frequently may climb to a high degree within a very short time. The sudden increase in temperature may not give the baby's caretaker sufficient time to react and control the fever or to determine its cause, Large amplitude body temperature variations are not uncommon in babies and infants. The earliest determination of a fever in a baby could result in the timely prevention of serious health consequences, including death of the infant.

When a baby wearing an absorbent undergarment expels urine or feces, there is usually no immediate reaction. It is the subsequent discomfort that will cause a baby to cry and thus warn an adult caretaker about the situation. Alternatively, the adult caring for a baby must proactively detect an odor of feces or physically inspect, the absorbent undergarment for urine. It is therefore advantageous to continuously monitor a baby's temperature when possible.

An inherent weakness of the current state of the art to monitor a baby's temperature is that it requires a specific temperature recording device to be monitored at repeated intermittent intervals. Continuous temperature monitoring exists in hospital settings but often limit a baby's mobility and are used for a different purpose, most likely after the high fever health damage has been caused. Currently there are no embedded undergarment early-detection thermal indicators to notify and prevent the serious consequences of an uncontrolled high fever. Currently, each of these issues must be addressed individually often times causing great confusion and frustration to a baby's caretaker.

### SUMMARY OF THE INVENTION

In one embodiment of the present inventive absorbent undergarment thermal indicator system consists of a combination of a thermometer integrated with an absorbent undergarment. The novel thermal indicator-absorbent undergarment combination can be custom sized so as to be integrated in selected sites of the absorbent undergarment to be comfortable, unobtrusive, visible, and in continuous contact with the underlying body of the user when worn and which readily displays the indicated temperature. The preferred location for the thermal indicator display on the absorbent undergarment is on the abdominal area of the user such that it usually maintains continuous contact with the body. Other possible body contact locations are possible, but are not limited to, as the sides, the back, and the gluteus. If continuous physical contact with the user's body can be assured, then any location on the undergarment is acceptable. The absorbent undergarment thermal indicator system may be used to display and/or to relay the temperature of the absorbent undergarment user's body via wireless transmission capabilities by one or more transmissions methods widely known to a person skilled in the art. The temperature of the absorbent undergarment user's body roughly corresponds to the skin temperature immediately beneath the contact site. Since different parts of the user's body may register temperature variations vis a vis known reliable body measurements, such as under the tongue or rectal locations, it is possible to preset the thermal indicator to account for such known differences according to the body location that it is expected to come in contact. The absorbent undergarment with an integrated thermal indicator is able to provide continuous temperature monitoring of the absorbent undergarment's user when continuous observation is desired.

### DRAWINGS

FIG. 1 is a top view of an absorbent undergarment integrated with a thermal indicating device, a visual temperature reading display, a urine sensor, a fecal matter sensor, and a wireless transmission device.

FIG. 2 is a front view of a user wearing an absorbent undergarment integrated with a thermal indicating device, a visual temperature reading display, a urine sensor, a fecal matter sensor, and a wireless transmission device.

### DETAILED DESCRIPTION OF THE INVENTION

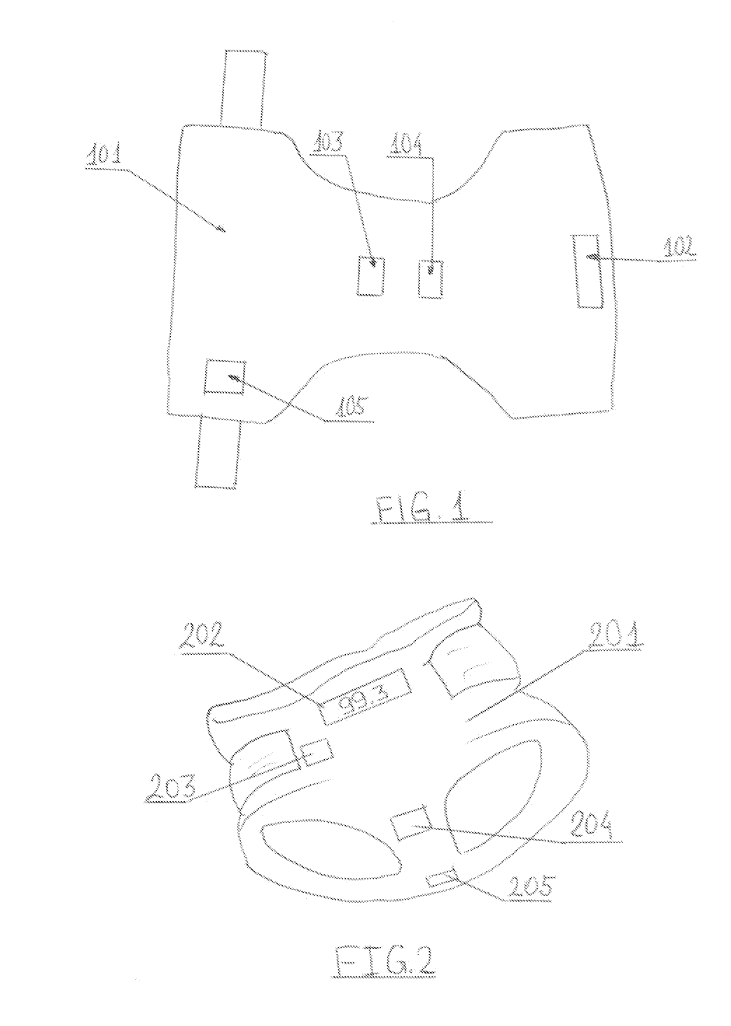

FIG. 1 depicts a top down view of a preferred embodiment of the invention.

The absorbent undergarment 101 is integrated at a selected location with a thermal indicating device 102. The absorbent undergarment 101 may consist of any number of conventionally suitable materials including, but not limited to woven cotton, linen, or other natural cloth or synthetic fabrics so long as the chosen material allows for integration with the thermal indicating device 102.

The thermal indicating device 102 may be any device suitable to give an accurate body temperature reading through direct contact with the user's body. The thermal indicating device 102 may be a mercury thermometer, infrared thermometer, or any other temperature measuring device known to a person skilled in the art. In the preferred embodiment, the thermal indicating device 102 is a liquid crystal thermochromic strip positioned at the front of the absorbent undergarment 101 so that it is in direct contact with the user's abdominal region. A liquid crystal thermochromic strip indicator has many qualities that are suitable for use with infants, such as safety, flexibility, low cost, easiness of manufacture, and possesses a relatively long shelf life allowing it to survive the duration of the absorbent undergarment's use. The liquid crystal thermochromic strip indicator is also UV ray and humidity resistant making the absorbent undergarment-thermal indicator integration practical and cost effective.

A visual temperature display device, described and depicted in FIG. 2, may be, but need not necessarily be, located in the same position as the thermal indicating device 102.

In an enhanced embodiment, the absorbent undergarment 101 may also have wireless transmission capabilities 105. Currently, many people utilize “baby monitors” to transmit any noise or crying in a baby's location to another room nearby. In a similar fashion, the preferred embodiment of the present invention can utilize wireless transmission capabilities 105 that would allow one to monitor a baby's temperature from a remote location, such as, but not limited to, a baby monitor, a remote computer, or handheld wireless device. The wireless transmission capabilities 105 may consist of any safe and cost effective means to transmit a wireless signal, as it is known to persons skilled in the art, including, but not limited to, RFID (radio frequency identification tag), Bluetooth, Wi-Fi, infrared, or modulated radio frequency.

In an alternative embodiment of the present invention, a thermal indicator 102, a urine sensor 104, and a feces sensor 103 are integrated within the absorbent undergarment 101 in such a manner as to most effectively detect when the user has soiled the garment. The urine sensor 104, feces sensor 103 and thermal indicating device 102 may be independent or a combination sensor and may one or all be connected to the wireless transmission capability 105 allowing one monitoring the absorbent undergarment user to better determine the cause of any discomfort experienced by the user.

FIG. 2 depicts a front view of an absorbent undergarment 201 in a deploy position as may be found on a user. FIG. 2 further depicts the preferred embodiment where the thermal indicating device showing a temperature display device 202 is integrated into the front of the undergarment so as to remain in contact with the body in the user's abdominal region.

The visual temperature display device 202 is capable of displaying the temperature recorded by the thermal indicating device 102 in multiple ways including through the use of alphanumeric characters, graphics, thermochromic graphics, and thermochromic alphanumeric characters. In the preferred embodiment shown, the temperature display device 202 is located opposite to the thermal indicating device 102 such that both devices are positioned at the front of the absorbent undergarment 201. This preferred positioning, as shown in FIG. 1, allows the individual monitoring the baby to view the visual temperature display device every time the baby is held in front to monitor and observe the baby's demeanor and facial expressions.

In this view of the alternative embodiment of the present invention, a urine sensor 204, and a feces sensor 205 are integrated within the absorbent undergarment 201 in such a manner as to most effectively detect when the user has soiled the garment. The urine sensor 204, feces sensor 205 and thermal display device 202 may be independent or a combination device and may one or all be connected to the wireless transmission capability 203 allowing one monitoring the absorbent undergarment user to better determine the cause of any discomfort experienced by the user.

What I claim is:

1. A body temperature monitoring device, the device comprising:
an absorbent undergarment worn by a user incapable of controlling their bladder or bowel movements, or are unable or unwilling to use a toilet;
at least one thermal indicator in direct contact with the user's body; and
at least one visual temperature display device capable of being viewed remotely from the absorbent undergarment

2. The body temperature monitoring device of claim 1, wherein the at least one thermal indicator is selected from the group consisting of: liquid crystal, mercury, and infrared measuring devices.

3. The body temperature monitoring device of claim 1, wherein the visual temperature display device consists of at least one selected from a group consisting of: alphanumeric characters, graphics, thermochromic graphics, and thermochromic alphanumeric characters.

4. The body temperature monitoring device of claim 1, wherein the visual temperature display device has wireless transmission capabilities.

5. The wireless transmission capabilities of claim 4 wherein the capabilities include at least one selected from the group consisting of: radio frequency identification tag, Bluetooth, Wi-Fi, infrared, and modulated radio frequency.

6. A body temperature monitoring device, the device comprising:
an absorbent undergarment worn by a user incapable of controlling their bladder or bowel movements, or are unable or unwilling to use a toilet;
at least one thermal indicator in direct contact with the user's body;
at least one visual temperature display device capable of being viewed remotely from the absorbent garment;
at least one urine sensor; and
at least one feces sensor.

7. The body temperature monitoring device of claim 6, wherein the at least one thermal indicator is selected from the group consisting of: liquid crystal, mercury and infrared measuring devices.

8. The body temperature monitoring device of claim 6, wherein the visual temperature display device consists of at least one selected from the group consisting of:
alphanumeric characters, graphics, thermochromic graphics, and thermochromic alphanumeric characters.

9. The body temperature monitoring device of claim 6, wherein the visual temperature display device has wireless transmission capabilities.

10. The wireless transmission capabilities of claim 9, wherein the capabilities include at least one selected from the group consisting of: radio frequency identification tag, Bluetooth, Wi-Fi, infrared, and modulated radio frequency.
